(19) United States
(12) Patent Application Publication
(10) Pub. No.: US 2002/0006404 A1
(43) Pub. Date: Jan. 17, 2002

(54) Treatment of cell malignancies using combination of B cell depleting antibody and immune modulating antibody related applications

(75) Inventors: Nabil Hanna, Rancho Santa Fe, CA (US); Kandasamy Hariharan, San Diego, CA (US)

Correspondence Address: PILLSBURY WINTHROP LLP, 1600 TYSONS BOULEVARD, MCLEAN, VA, 22102, US

(73) Assignee: IDEC Pharmaceuticals Corporation

(21) Appl. No.: 09/772,938

(22) Filed: Jan. 31, 2001

Related U.S. Application Data
(63) Continuation-in-part of application No. 09/435,992, filed on Nov. 8, 1999.

(51) Int. Cl. A61K 39/395

(52) U.S. Cl. 424/142.1; 424/155.1

(57) ABSTRACT

A combination antibody therapy for treating B cell malignancies using an immunoregulatory antibody, especially an anti-B7, anti-CD23, or anti-CD40L antibody and a B cell depleting antibody, especially anti-CD19, anti-CD20, anti-CD22 or anti-CD37 antibody is provided.

### CROSS REFERENCE TO RELATED APPLICATION

This application is a continuation-in-part of U.S. Ser. No. 09/435,992 filed Nov. 8, 1999 incorporated by reference in its entirety thereon.

### FIELD OF THE INVENTION

The invention relates to a synergistic combination antibody therapy for treatment of B cell malignancies, especially B cell lymphomas and leukemias. This synergistic antibody combination comprises at least one antibody having substantial B cell depleting activity (e.g., an anti-CD19, CD20, CD22 or CD37 antibody) and an antibody that modulates or regulates the immune system, e.g., by modulating B cell/T cell interactions and/or B cell activity, differentiation or proliferation (e.g., anti-B7, anti-CD40, anti-CD23 or anti-CD40L).

### BACKGROUND OF INVENTION

It is known that B cell malignancies, e.g., B cell lymphomas and leukemias may be successfully treated using antibodies specific to B cell antigens that possess B cell depleting activity. Examples of B cell antibodies that have been reported to possess actual or potential application for the treatment of B cell malignancies include antibodies specific to CD20, CD19, CD22, and CD37.

In fact, a chimeric anti-CD20 antibody, RITUXAN® (also known as Rixtimab, MabThera®, IDEC-C2B8 and C2B) is the first FDA approved monoclonal antibody for treatment of cancer (non-Hodgkin's lymphoma) and was developed by IDEC Pharmaceuticals Corporation (see U.S. Pat. Nos. 5,843,439; 5,776,456; and 5,736,137).

Also, the use anti-CD37 antibodies having B cell depleting activity have been well reported to possess potential for treatment of B cell lymphoma. See e.g., Presr et al., J. Clin. Oncol. 7(8): 1027-1038 (August 1989); Grossbard et al., Blood 8(4): 863-876 (Aug. 15, 1992).

Further, the use of B cell antibodies specific to CD22 for treatment of B cell malignancies has been reported. For example, an unlabelled antibody that binds CD22, Lymphocide® is now in clinical trials for treatment of indolent non-Hodgkin's lymphoma. Also, an yttrium 90 labeled form of the same antibody is being clinically investigated for treatment of indolent and aggressive non-Hodgkin's lymphoma.

Still further, the potential use of anti-CD 19 antibodies for treatment of B cell malignancies has been reported.

Also, the treatment of B cell malignancies using immunoregulatory or immunomodulatory antibodies has been suggested. For example, it has been reported that anti-CD40 antibody administration to mice with human B cell lymphoma xenografts enhanced their survival (see Funakushi et al., Blood 83: 2787-2797 (1994), Murphy et al., Blood 86: 1946-1953 (1995) and Tutt et al., J. Immunol. 161: 3176-3185 (1998)). Also, CD40 signaling has been suggested to interact with CD20 (Ledbetter et al., Circ. Shock 44: 67-72 (1999)).

In has further been suggested that CD40L may play a role in cell contact-dependent interaction of tissue B cells (CD40⁺) within neoplastic follicles or Reed-Sternberg cells (CD40⁺) in Hodgkin's disease areas (Carbone et al., Am. J. Pathol. 147: 912-922 (1995)).

Still further, the use of anti-B7 antibodies for treatment of B cell lymphoma was mentioned in a patent assigned to IDEC Pharmaceuticals Corporation. However, the focus of the patent was the use thereof for treating diseases which immunosuppression is therapeutically beneficial. Examples included allergic, autoimmune and transplant indications. Also mentioned was the use of the discussed anti-B7 antibodies for treatment of B cell lymphoma. (U.S. Pat. No. 6,113,198).

Thus, based on the foregoing, it is clear that numerous antibodies have been reported to possess therapeutic potential for treatment of B cell malignancies. Notwithstanding this fact, it is an object of the invention to provide novel antibody regimens for treatment of B cell lymphoma.

### BRIEF DESCRIPTION AND OBJECTS OF THE INVENTION

Toward that end, it is an object of the invention to provide a novel improved antibody therapy for treatment of B cell malignancies.

More specifically, it is an object of the invention to provide a novel antibody regimen for treatment of a B cell malignancy involving the administration of at least one B cell depleting antibody and at least one immunoregulatory or immunomodulatory antibody.

Even more specifically, it is an object of the invention to provide a novel antibody therapy for treatment of B cell malignancies that involves the administration of at least one B cell depleting antibody selected from an anti-CD20, anti-CD19, anti-CD22 or anti-CD37 antibody and at least one immunomodulatory antibody selected from an anti-B7, anti-CD23, anti-CD40, anti-CD40L or anti-CD4 antibody.

It is another object of the invention to provide a novel therapeutic regimen for treatment of a B cell malignancy such as non-Hodgkin's lymphoma or chronic lymphocyte leukemia (CLL) by the administration of an antibody to CD20 (preferably RITUXAN®) and an antibody to B7 or CD40L (respectively preferably Primatized anti-B7 antibodies reported in U.S. Pat. No. 6,113,198 to Anderson et al, or humanized anti-CD40L antibody reported in U.S. Pat. No. 6,001,358, assigned to IDEC Pharmaceuticals Corporation.

It is another object of the invention to provide novel compositions and kits for treatment of B cell malignancies, in B cell lymphomas and leukemias, that include at least one immunoregulatory or immunomodulatory antibody and at least one B cell depleting antibody. Preferably, the immunoregulatory or immunomodulatory antibody will comprise an anti-CD40, anti-CD40L or anti-B7 antibody and the B cell depleting antibody will be specific to CD20, CD19, CD22 or CD37. Most preferably, the composition will comprise an anti-CD40L or anti-B7 antibody and an anti-CD20 antibody.

### BRIEF DESCRIPTION OF THE FIGURES

FIG. 1. Sensitivity of B-lymphoma cells to adriamycin after 4 hour exposure.

FIG. 2. (Panel A) Anti CD40L (IDEC-131) overrides CD40L mediated resistance to killing by ADM of B-lymphoma cells. (Panel B) Effect of RITUXAN® on normal and sCD40L pre-treated DHL-4 cells.

FIG. 3. (Panel A) Blocking of CD40L mediated cell survival of B-CLL by anti-CD40L antibody (IDEC-131). (Panel B) Blocking of CD40L mediated survival of B-CLL by Idec's C2B8.

FIG. 4. FACS analysis comprising HLA-DR expression in CD19⁺ CLL cells cultured with sCD40L and not cultured with sCD40L.

### DETAILED DESCRIPTION OF THE INVENTION

The present invention provides a novel combination antibody regimen that involves the administration of at least one immunoregulatory or immunomodulatory antibody, e.g., an anti-B7 or anti-CD40 or anti-CD40L antibody and at least one B cell depleting antibody, e.g., an anti-CD20, anti-CD19, anti-CD22 or anti-CD37 antibody having substantial B cell depleting activity.

It is believed that such combination will afford synergistic results based on the different mechanisms by which the antibodies elicit a therapeutic benefit. In particular, it is theorized that the complementary mechanisms of action will yield a more durable and potent clinical response as it is believed that the B cell depleting antibody will deplete activated B cells which may be resistant to the action of immunoregulatory or immunomodulatory antibodies such as anti-B7 or anti-CD40L antibodies. Such activated B cells can otherwise serve as effective antigen presenting cells for T cells as well as antibody producing cells. In the context of B cell malignancies, such activated B cells may include malignant cells which unless eradicated by give rise to new cancer cells and tumors.

Prior to discussing the invention, the following definitions are provided:

“B Cell Depleting Antibody” therein is an antibody or fragment that upon administration, results in demonstrable B cell depletion. Preferably, such antibody, after administration, typically within about several days or less, will result in a depletion of B cell number by about 50% or more. In a preferred embodiment, the B cell depleting antibody will be RITUXAN® (a chimeric anti-CD20 antibody) or one having substantially the same or at least 20-50% the cell depleting activity of RITUNAN®. “Immunoregulatory Antibody” refers to an antibody that elicits an effect on the immune system by a mechanism different from B cell depletion, e.g., by CDL and/or ADCC activity. Examples of such include antibodies that inhibit T cell immunity, B cell immunity, e.g. by inducing tolerance (anti-CD40L, anti-CD40) or other immunosuppressant antibodies, e.g., those that inhibit B7 cell signaling (anti-B7.1, anti-B7.2, anti-CD4, anti-CD23, etc.). In some instances, the immunoregulatory antibody may possess the ability to potientiate apoptosis.

A “B cell surface marker” herein is an antigen expressed on the surface of a B cell which can be targeted with an antagonist which binds thereto. Exemplary B cell surface markers include the CD10, CD19, CD20, CD21, CD22, CD23, CD24, CD37, CD53, CD72, CD73, CD74, CDw75, CDw76, CD77, CDw78, CD79a, CD79b, CD80 (B7.1), CD81, CD82, CD83, CDw84, CD85 and CD86 (B7.2) leukocyte surface markers. The B cell surface marker of particular interest is preferentially expressed on B cells compared to other non-B cell tissues of a mammal and may be expressed on both precursor B cells and mature B cells. In one embodiment, the marker is one, like CD20 or CD 19, which is found on B cells throughout differentiation of the lineage from the stem cell stage up to a point just prior to terminal differentiation into plasma cells. The preferred B cell surface markers herein are CD 19, CD20, CD23, CD80 and CD86.

The “CD20” antigen is a −35 kDa, non-glycosylated phosphoprotein found on the surface of greater than 90% of B cells from peripheral blood or lymphoid organs. CD20 is expressed during early pre-B cell development and remains until plasma cell differentiation. CD20 is present on both normal B cells as well as malignant B cells. Other names for CD20 in the literature include “B-lymphocyte-restricted antigen” and “Bp35”. The CD20 antigen is described in Clark et al. PNAS (USA) 82:1766(1985).

The “CD 19” antigen refers to a −90 kDa antigen identified, for example, by the HD237-CD19 or B4 antibody (Kiesel et al. Leukemia Research II, 12: 1119 (1987)). Like CD20, CD19 is found on cells throughout differentiation of the lineage from the stem cell stage up to a point just prior to terminal differentiation into plasma cells. Binding of an antagonist to CD 19 may cause internalization of the CD 19 antigen.

The “CD22” antigen refers to an antigen expressed on B cells, also known as “BL-CAM” and “LybB” that is involved in B cell signaling and an adhesion. (See Nitschke et al., Curr. Biol 7:133 (1997); Stamenkovic et al., Nature 345:74 (1990)). This antigen is a membrane immunoglobulin-associated antigen that is tyrosine phosphorylated when membrane Ig is ligated. (Engel et al., J. Etyp. Med. 181(4):1521 1586 (1995)). The gene encoding this antigen has been cloned, and its lg domains characterized.

B7 antigen includes the B7.1 (CD80), B7.2 (CD81) and B7.3 antigen, which are transmembrane antigens expressed on B cells. Antibodies which specifically bind B7 antigens, including human B7.1 and B7.2 antigens are known in the art. Preferred B7 antibodies comprise the primatized® B7 antibodies disclosed by Anderson et al. in U.S. Pat. No. 6,113,198, assigned to IDEC Pharmaceuticals Corporation, as well as human and humanized B7 antibodies.

CD23 refers to the low affinity receptor for IgE expressed by B and other cells. In the present invention, CD23 will preferably be human CD23 antigen. CD23 antibodies are also known in the art. Most preferably, in the present invention, the CD23 antibody will be a human or chimeric anti-human CD23 antibody comprising human IgGI or IgG3 constant domains.

A B cell “antagonist” is a molecule which, upon binding to a B cell surface marker, destroys or depletes B cells in a mammal and/or interferes with one or more B cell functions, e.g. by reducing or preventing a humoral response elicited by the B cell. The antagonist preferably is able to deplete B cells (i.e. reduce circulating B cell levels) in a mammal treated therewith. Such depletion may be achieved via various mechanisms such antibody-dependent cell-mediated cytotoxicity (ADCC) and/or complement dependent cytotoxicity (CDC), inhibition of B cell proliferation and/or induction of B cell death (e.g. via apoptosis). Antagonists included within the scope of the present invention include antibodies, synthetic or native sequence peptides and small molecule antagonists which bind to the B cell marker, optionally conjugated with or fused to a cytotoxic agent.

A CD40L antagonist is a molecule that specifically binds CD40L and preferably antagonizes the interaction of CD40L and CD40. Examples thereof include antibodies and antibody fragments that specifically bind CD40L, soluble CD40, soluble CD40 fusion proteins, and small molecules that bind CD40L. The preferred antagonist according to the invention comprises an antibody or antibody fragment specific to CD40.

“Antibody-dependent cell-mediated cytotoxicity” and “ADCC” refer to a cell mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcyRIII only, whereas monocytes express FcyRI, FcyRII and FcyRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector functions. Preferably, the cells express at least FcyRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcyRI, FcyRII, and FcyRII subclasses, including allelic variants and alternatively spliced forms of these receptors. FcyRII receptors include FcyRIIA (an “activating receptor”) and FcyRUB (an “inhibiting receptor”), which have similar amino acid sequences that differ primarily in the cytoplasmic domains thereof. Activating receptor FcyRIIA contains an immunoreceptor tyrosine-based activation motif (ITAM) in its cytoplasmic domain. Inhibiting receptor FcyRIIB contains an immunoreceptor tyrosine-based inhibition motif (ITIM) in its cytoplasmic domain. (see review M. in Daeon, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al, J. Immunol. 117:587 (1976) and Kim et al., J. Immunol. 24:249 (1994)).

“Complement dependent cytotoxicity” or “CDC” refers to the ability of a molecule to lyse a target in the presence of complement. The complement activation pathway is initiated by the binding of the first component of the complement system (Clq) to a molecule (e.g. an antibody) complexed with a cognate antigen. To assess complement activation, a CDC assay, e.g. as described in Gazzano-Santoro et al., J. Immunol. Methods 202:163 (1996), may be performed.

“Growth inhibitory” antagonists are those which prevent or reduce proliferation of a cell expressing an antigen to which the antagonist binds. For example, the antagonist may prevent or reduce proliferation of B cells in vitro and/or in vivo.

Antagonists which “induce apoptosis” are those which induce programmed cell death, e.g. of a B cell, as determined by binding of annexin V, fragmentation of DNA, cell shrinkage, dilation of endoplasmic reticulum, cell fragmentation, and/or formation of membrane vesicles (called apoptotic bodies).

The term “antibody” herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity.

“Antibody fragments” comprise a portion of an intact antibody, preferably comprising the antigen-binding or variable region thereof. Examples of antibody fragments include Fab, Fab′, F(ab′)Z, and Fv fragments; diabodies; linear antibodies; single-chain antibody molecules; and multispecific antibodies formed from antibody fragments.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a 13-sheet configuration, connected by three hypervariable regions, which form loops connecting, and in some cases forming part of, the B-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, MD. (1991)). The constant domains are not involved directly in binding an antibody to an antigen, but exhibit various effector functions, such as participation of the antibody in antibody dependent cellular cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CHI) of the heavy chain. Fab′ fragments differ from Fab fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear at least one free thiol group. F(ab′)Z antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgGI, IgG2, IgG3, IgG4, IgA, and IgA2. The heavy-chain constant domains that correspond to the different classes of antibodies are called alpha, delta, epsilon, gamma and mu, respectively. Preferably, the heavy-chain constant domains will complete the gamma-1, gamma-2, gamma-3 and gamma-4 constant region. Preferably, these constant domains will also comprise modifications to enhance antibody stability such as the P and E modification disclosed in U.S. Pat. No. 6,011,138 incorporated by reference in its entirety herein. The subunit structures and three dimensional configurations of different classes of immunoglobulins are well known.

“Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chains) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc) and human constant region sequences.

“Humanized” forms of non-human (e.g., marine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al, Nature 321:522-525 (1986); Riechmann et al., Nature 332:323-329 (1988); and Presta, Curr. Op. Struct. Biol. 2:593-596 (1992).

The term “hypervariable region” when used herein refers to the amino acid residues of an antibody which are responsible for antigen-binding. The hypervariable region comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (LI), 50-52 (L2) and 91-96 (L3) in the light chain variable domain and 26-32 (Hi), 53-55 (H2) and 96-101 (H3) in the heavy chain variable domain; Chothia and Lesk.l. Mol. Biol. 196:901-917 (1987)). “Framework” or “FR” residues are those variable domain residues other than the hypervariable region residues as herein defined.

An antagonist “which binds” an antigen of interest, e.g. a B cell surface marker, is one capable of binding that antigen with sufficient affinity such that the antagonist is useful as a therapeutic agent for targeting a cell, i.e. a B cell, expressing the antigen.

An “anti-CD20 antibody” herein is an antibody that specifically binds CD20 antigen, preferably human CD20, having measurable B cell depleting activity, preferably having at least about 10% the B cell depleting activity of RITUXAN® (see U.S. Pat. No. 5,736,137, incorporated by reference herein in its entirety).

An “anti-CD22 antibody” herein is an antibody that specifically binds CD22 antigen, preferably human CD22, having measurable B cell depleting activity, preferably having at least about 10% the B cell depleting activity of RITUXAN® (see U.S. Pat. No. 5,736,137, incorporated by reference herein in its entirety).

An “anti-CD 19 antibody” herein is an antibody that specifically binds CD 19 antigen, preferably human CD 19, having measurable B cell depleting activity, preferably having at least about 10% the B cell depleting activity of RITUXAN® (see U.S. Pat. No. 5,736,137, incorporated by reference herein in its entirety).

An “anti-CD37 antibody” herein is an antibody that specifically binds CD37 antigen, preferably human CD37, having measurable B cell depleting activity, preferably having at least about 10% the B cell depleting activity of RITUXAN® (see U.S. Pat. No. 5,736,137, incorporated by reference herein in its entirety).

An “anti-B7 antibody” herein is an antibody that specifically binds B7.1, B7.2 or B7.3, most preferably human B7.3, that inhibits B7/CD28 interactions and, which more does not substantially inhibit B7/CTLA-4 interactions, and even more preferably, the particular antibodies described in U.S. Pat. No. 6,113,898, incorporated by reference in its entirety herein. It has recently been shown that these antibodies promote apoptosis. Therefore, they are well suited for anti-neoplastic applications.

An “anti-CD40L antibody” is an antibody that specifically binds CD40L (also known as CD154, gp39, TBAM), preferably one having agonistic activity. A preferred anti-Cd40L antibody is one having the specificity of a humanized antibody disclosed in U.S. Pat. No. 6,011,358 (assigned to IDEC Pharmaceuticals Corporation), incorporated by reference in its entirety herein.

An “anti-CD4 antibody” is one that specifically binds CD4, preferably human CD4, more preferably a primatized or humanized anti-CD4 antibody.

An “anti-CD40 antibody” is an antibody that specifically binds CD40, preferably human CD40, such as those disclosed in U.S. Pat. No. 5,874,085, 5,874,082, 5,801,227, 5,674,442, snf 5,667,165, all of which are incorporated by reference herein.

Preferably, both the B cell depleting antibody and the immunoregulatory antibody will contain human constant domains. Suitable antibodies may include IgGI, IgG2, IgG3 and IgG4 isotypes.

Specific examples of antibodies which bind the CD20 antigen include: “Rituximab” (“RITUXAN®”) (U.S. Pat. No. 5,736,137, expressly incorporated herein by reference); yttrium-[90]-labeled 2B8 murine antibody “Y2B8” (U.S. Pat. No. 5,736,B7, expressly incorporated herein by reference); murine IgG2a “B 1” optionally labeled with 1311, <<1311 B1” antibody (BEXXARTM) (U.S. Pat. No. 5,595,721, expressly incorporated herein by reference); murine monoclonal antibody “1F5” (Press et al. Blood 69(2):584-591 (1987); and “chimeric 2H7” antibody (U.S. Pat. No. 5,677,180, expressly incorporated herein by reference).

Specific examples of antibodies which bind CD22 include LymphocideTM reported by Immuno-medics, now in clinical trials for non-Hodgkin's lymphoma. Examples of antibodies that bind B7 antigen include the B7 antibody reported U.S. Pat. No. 5,885,577, issued to Linsley et al, the anti-B7 antibody reported in U.S. Pat. No. 5,869,050, issued in DeBoer et al, assigned to Chiron Corporation, and the primatized® anti-B7 antibody disclosed in U.S. Pat. No. 6,113,198 to Anderson et al., all of which are incorporated by reference in their entirety.

Specific examples of antibodies that bind CD23 are well known and preferably include the primatized® antibodies specific to human CD23 reported by Reff et al., in U.S. Pat. No. 6,011,138, issued on Jul. 4, 1999, co-assigned to IDEC Pharmaceuticals Corp. and Seikakagu Corporation of Japan; those reported by Bonnefoy et al., No. 96 12741; Rector et al. J. Immunol. 55:481-488 (1985); Flores-Rumeo et al. Science 241:1038-1046 (1993); Sherr et al. J. Immunol., 142:481-489 (1989); and Pene et al., PNAS, USA 85:6820-6824 (1988). Such antibodies are reportedly useful for treatment of allergy, autoimmune diseases, and inflammatory diseases.

The terms “rituximab” or “RITUXAN®” herein refer to the genetically engineered chimeric murine/human monoclonal antibody directed against the CD20 antigen and designated “C2B8” in U.S. Pat. No. 5,736,B7, expressly incorporated herein by reference. The antibody is an IgGI kappa immunoglobulin containing murine light and heavy chain variable region sequences and human constant region sequences. Rituximab has a binding affinity for the CD20 antigen of approximately 8.0 nM.

An “isolated” antagonist is one which has been identified and separated and/or recovered from a component of its natural environment. Contaminant components of its natural environment are materials which would interfere with diagnostic or therapeutic uses for the antagonist, and may include enzymes, hormones, and other proteinaceous or nonproteinaceous solutes. In preferred embodiments, the antagonist will be purified (1) to greater than 95% by eight of antagonist as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antagonist includes the antagonist in situ within recombinant cells since at least one component of the antagonist's natural environment will not be present. Ordinarily, however, isolated antagonist will be prepared by at least one purification step.

“Mammal” for purposes of treatment refers to any animal classified as a mammal, including humans, domestic and farm animals, and zoo, sports, or pet animals, such as dogs, horses, cats, cows, etc. Preferably, the mammal is human.

“Treatment” refers to both therapeutic treatment and prophylactic or preventative measures. Those in need of treatment include those already with the disease or disorder as well as those in which the disease or disorder is to be prevented. Hence, the mammal may have been diagnosed as having the disease or disorder or may be predisposed or susceptible to the disease.

B Cell Malignancy

According to the present invention this includes any B cell malignancy, e.g., B cell lymphomas and leukemias. Preferred examples include Hodgkin's disease (all forms, e.g., relapsed Hodgkin's disease, resistant Hodgkin's disease) non-Hodgkin's lymphomas (low grade, intermediate grade, high grade, and other types). Examples include small lymphocytic/B cell chronic lymphocytic leukemia (SLL/B-CLL), lymhoplasmacytoid lymphoma (LPL), mantle cell lymphoma (MCL), follicular lymphoma (FL), diffuse large cell lymphoma (DLCL), Burkitt's lymphoma (BL), AIDS-related lymphomas, monocytic B cell lymphoma, angioimmunoblastic lymphoadenopathy, small lymphocytic, follicular, diffuse large cell, diffuse small cleaved cell, large cell immunoblastic lymphoblastoma, small, non-cleaved, Burkitt's and non-Burkitt's, follicular, predominantly large cell; follicular, predominantly small cleaved cell; and follicular, mixed small cleaved and large cell lymphomas. See, Gaidono et al., “Lymphomas”, IN CANCER: PRINCIPLES & PRACTICE OF ONCOLOGY, Vol. 2: 2131-2145 (DeVita et al., eds., 5^{th} ed. 1997).

Other types of lymphoma classifications include immunocytomal Waldenstrom's MALT-type/monocytoid B cell, mantle cell lymphoma B-CLL/SLL, diffuse large B-cell lymphoma, follicular lymphoma, and precursor B-LBL.

As noted B cell malignancies further includes especially leukemias such as ALL-L3 (Burkitt's type leukemia), chronic lymphocytic leukemia (CLL) and monocytic cell leukemias.

The expression “therapeutically effective amount” refers to an amount of the antagonist which is effective for preventing, ameliorating or treating the Ball malignancy disease in question.

The term “immunosuppressive agent” as used herein for adjunct therapy refers to substances that act to suppress or mask the immune system of the mammal being treated herein. This would include substances that suppress cytokine production, downregulate or suppress self-antigen expression, or mask the MHC antigens. Examples of such agents include 2-amino-6-aryl-5-substituted pyrimidines (see U.S. Pat. No. 4,665,077, the disclosure of which is incorporated herein by reference), azathioprine; cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as glucocorticosteroids, e.g., prednisone, methylprednisolone, and dexamethasone; cytokine or cytokine receptor antagonists including anti-interferon-α, β- or δ-antibodies, anti-tumor necrosis factor-α antibodies, anti-tumor necrosis factor-β antibodies, anti-interleukin-2 antibodies and anti-IL-2 receptor antibodies; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published 7/26/90), streptolanase; TGF-β; streptodomase; RNA or DNA from the host; FK506; RS-61443; deoxyspergualin; rapamycin; T-cell receptor (Cohen et al., U.S. Pat. No. 5,114,721); T-cell receptor fragments (Offner et al., Science, 251: 430-432 (1991); WO 90/11294; laneway, Nature, 341: 482 (1989); and WO 91/01133); and T cell receptor antibodies (EP 340,109) such as T10B9.

The term “cytotoxic agent” as used herein refers to a substance that inhibits or prevents the function of cells and/or causes destruction of cells. The term is intended to include radioactive isotopes (e.g. At211 1131 1125 Y9o Re 186 Re 1g8 sM153 Bi212 p32 and radioactive isotopes of Lu), chemotherapeutic agents, and toxins such as small molecule toxins or enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and pip osulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime nitrogen mustards such as chiorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, NJ) and doxetaxel (Taxotere, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT 11; topoisomerase inhibitor RFS 2000; difluoromethylomithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and antiandrogens such as flutamide, nilutamide, bicalutamide, leuprolide, and goserelin; and pharmaceutically acceptable salts, acids or derivatives of any of the above.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-13; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocytemacrophage-CSF (GM-CSF); and granulocyte-CSF (G-CSF); interleukins (ILs) such as IL-1, IL-1a, IL-2, IL-g, IL-4, IL-5, IL-6, IL-7, IL-8, IL-9, IL-11, IL-12, IL-15; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp.375-382, 615th Meeting Belfast (1986) and Stella et al., “Prodrugs: A Chemical Approach to Targeted Drug Delivery,” Directed Drug Delivery, Borchardt et al., (ed.), pp. 247-267,Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs,13-lactam-containing prodrugs, optionally substituted phenoxyacetamide-containing prodrugs or optionally substituted phenylacetamide-containing prodrugs, 5-fluorocytosine and other 5fluorouridine prodrugs which can be converted into the more active cytotoxic free drug. Examples of cytotoxic drugs that can be derivatized into a prodrug form for use in this invention include, but are not limited to, those chemotherapeutic agents described above.

A “liposome” is a small vesicle composed of various types of lipids, phospholipids and/or surfactant which is useful for delivery of a drug (such as the antagonists disclosed herein and, optionally, a chemotherapeutic agent) to a mammal. The components of the liposome are commonly arranged in a bilayer formation, similar to the lipid arrangement of biological membranes.

The term “package insert” is used to refer to instructions customarily included in commercial packages of therapeutic products, that contain information about the indications, usage, dosage, administration, contraindications and/or warnings concerning the use of such therapeutic products.

II. Production of Antibodies

The methods and articles of manufacture of the present invention use, or incorporate, an antibody that has immunoregulatory activity, e.g. anti-B7, anti-CD23, anti-CD40L, anti-CD4 or anti-CD40 antibody, and an antibody that binds to a B cell surface marker having B depleting activity, e.g., anti-CD20, anti-CD22, anti-CD 19, or anti-CD37 antibody. Accordingly, methods for generating such antibodies will be described herein.

The molecule to be used for production of, or screening for, antigen(s) may be, e.g., a soluble form of the antigen or a portion thereof, containing the desired epitope. Alternatively, or additionally, cells expressing the antigen at their cell surface can be used to generate, or screen for, antagonist(s). Other forms of the B cell surface marker useful for generating antagonists will be apparent to those skilled in the art. Suitable antigen sources for CD40L, CD40, CD19, CD20, CD22, CD23, CD37, CD4 and B7 antigen (e.g., B7.1, B7.2) antigen for producing antibodies according to the invention are well known.

Preferably, the CD40L antibody or anti-CD40L antibody will be the =humanized anti-CD40L antibody disclosed in U.S. Pat. No. 6,001,358, issued on Jun. 14, 1999, and assigned to IDEC Pharmaceuticals Corporation.

While a preferred CD40L antagonist is an antibody, antagonists other than antibodies may also be administered. For example, the antagonist may comprise soluble CD40, a CD40 fusion protein or a small molecule antagonist optionally fused to, or conjugated with, a cytotoxic agent (such as those described herein). Libraries of small molecules may be screened against the B cell surface marker of interest herein in order to identify a small molecule which binds to that antigen. The small molecule may further be screened for its antagonistic properties and/or conjugated with a cytotoxic agent.

The antagonist may also be a peptide generated by rational design or by phage display (WO98/35036 published Aug. 13, 1998), for example. In one embodiment, the molecule of choice may be a “CDR mimic” or antibody analogue designed based on the CDRs of an antibody, for example. While the peptide may be antagonistic by itself, the peptide may optionally be fused to a cytotoxic agent or to an immunoglobulin Fc region (e.g., so as to confer ADCC and/or CDC activity on the peptide).

Exemplary techniques for the production of the antibody antagonists used in accordance with the present invention are described.

(i) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succiic anhydride, SOC 1₂, or R¹N═C═NR, where R and R¹ are different alkyl groups.

Animals are immunized against the antigen, immunogenic conjugates, or derivatives by combining, e.g. 100 μg or 5 μg of the protein or conjugate (for rabbits or mice, respectively) with 3 volumes of Freund's complete adjuvant and injecting the solution intradermally at multiple sites. One month later the animals are boosted with 1/5 to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(ii) Monoclonal Antibodies

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp.59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Manassas, Va., USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the 30 Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp.59-103 (Academic Press, 1986)). Suitable culture media for this purpose include, for example, D-MEM or RPML-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Pluckthun, Immunol. Revs., 130:151-188 (1992).

In a further embodiment, antibodies or antibody fragments can be isolated from antibody phage libraries generated using the techniques described in McCafferty et al., Nature, 348:552-554(1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al, Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. ScL USA, 81:6851 (1984)), or by covalently joining to the immunoglobulin coding sequence all or part of the coding sequence for a non-immunoglobulin polypeptide.

Typically, such non-immunoglobulin polypeptides are substituted for the constant domains of an antibody, or they are substituted for the variable domains of one antigencombining site of an antibody to create a chimeric bivalent antibody comprising one antigen-combining site having specificity for an antigen and another antigen-combining site having specificity for a different antigen.

(iii) Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so called “best-fit” method, the sequence of the variable domain of a rodent antibody is screened against the entire library of known human variable-domain sequences. The human sequence which is closest to that of the rodent is then accepted as the human framework region (FR) for the humanized antibody (Suns et al., J. Immunol., 151:2296 (1993); Chothiaetal., J. Mol. Biol, 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

(iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region PH) gene in chimeric and germ-line mutant mice results in complete inhibition of endogenous antibody production. Transfer of the human germ-line immunoglobulin gene array in such germ line mutant mice will result in the production of human antibodies upon antigen challenge. See, e.g., Jakobovits et al., Proc. Mad. Acad. Sci. USA, 90:2551 (1993); Jakobovits et al., Nature, 362:255-258 (1993); Bruggermann et al., Year in immuno., 7:33 (1993); and U.S. Pat. Nos. 5,591,669, 5,589,369 and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self antigens) can be isolated essentially following the techniques described by Marks et al., J. Mol. Biol, 222:581-597 (1991), or Griffith et al., EMBO J. 12:725-734 (1993). See, also, U.S. Pat. Nos. 5,565,332 and 5,573,905.

Human antibodies may also be generated by in vitro activated B cells (see U.S. Pat. No. 5,567,610 and 5,229,275).

(v) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10: 163-167 (1992)). According to another approach, F(ab′)2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

(vi) Bispecific Antibodies

Bispecific antibodies are antibodies that have binding specificities for at least two different epitopes. Exemplary bispecific antibodies may bind to two different epitopes of the B cell surface marker. Other such antibodies may bind a first B cell marker and further bind a second B cell surface marker. Alternatively, an anti-B cell marker binding arm may be combined with an arm which binds to a triggering molecule on a leukocyte such as a T-cell receptor molecule (e.g. CD2 or CD3), or Fc receptors for IgG (FcyR), such as FcyRI (CD64), FcyRII (CD32) and FcyRIII (CD 16) so as to focus cellular defense mechanisms to the B cell. Bispecific antibodies may also be used to localize cytotoxic agents to the B cell. These antibodies possess a B cell marker-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab)2 bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CHI) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chains) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229:81(1985) describe a procedure wherein intact antibodies are proteolytically cleaved to generate F(ab′)Z fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et aL, J. Exp. Med., 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L})by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60(1991).

III. Conjugates and Other Modifications of the Antagonist

The antibodies used in the methods or included in the articles of manufacture herein are optionally conjugated to a cytotoxic agent.

Chemotherapeutic agents useful in the generation of such antibody-cytotoxic agent conjugates have been described above.

Conjugates of an antibody and one or more small molecule toxins, such as a calicheamicin, a maytansine (U.S. Pat. No. 5,208,020), a trichothene, and CC 1065 are also contemplated herein. In one preferred embodiment of the invention, the antagonist is conjugated to one or more maytansine molecules (e.g. about 1 to about 10 maytansine molecules per antagonist molecule). Maytansine may, for example, be converted to May SS-Me which may be reduced to May-SH3 and reacted with modified antagonist (Charm et al. Cancer Research 52:127-131(1992)) to generate a maytansinoid-antagonist conjugate.

Alternatively, the antibody may be conjugated to one or more calicheamicin molecules. The calicheamicin family of antibiotics are capable of producing double stranded DNA breaks at sub-picomolar concentrations. Structural analogues of calicheamicin which may be used include, but are not limited to, γ₁^{I}, α₂^{I}, α₃^{I}, N-acetyl-γ₁^{I}, PSAG and O^{I}₁, (Hinman et al. Cancer Research 53:3336-3342 (1993) and Lode et al, Cancer Research 58: 2925-2928 (1998)).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates antibody conjugated with a compound with nucleolytic activity (e.g. a ribonuclease or a DNA endonuclease such as a deoxyribonuclease; DNase).

A variety of radioactive isotopes are available for the production of radioconjugated antagonists. Examples include At²¹¹, I¹³¹, I¹²⁵, Y⁹⁰, Re¹⁸⁶, RE¹⁸⁸, Sm¹⁵³, Bi²¹², P³² and radioactive isotopes of Lu.

Conjugates of the antibody and cytotoxic agent may be made using a variety of bifunctional protein coupling agents such as N-succinimidyl-3-(2-pyriyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1 isothiocyanatobenzyl-3-methyldiethylene triaminepentaacetic.acid (MX-DTPA) is an exemplary chelating agent for conjugation of radionucleotide to the antagonist. See WO94/11026. The linker may be a “cleavable linker” facilitating release of the cytotoxic drug in the cell. For example, an acid-labile linker, peptidase-sensitive linker, dimethyl linker or disulfide-containing linker (Charm et al. Cancer Research 52:127-131 (1992)) may be used.

Alternatively, a fusion protein comprising the antibody and cytotoxic agent may be made, e.g. by recombinant techniques or peptide synthesis.

In yet another embodiment, the antibody may be conjugated to a “receptor” (such streptavidin) for utilization in tumor pretargeting wherein the antagonist-receptor conjugate is administered to the patient, followed by removal of unbound conjugate from the circulation using a clearing agent and then administration of a “ligand” (e.g. avidin) which is conjugated to a cytotoxic agent (e.g. a radionucleotide).

The antibodies of the present invention may also be conjugated with a prodrug activating enzyme which converts a prodrug (e.g. a peptidyl chemotherapeutic agent, see WO81/01145) to an active anti-cancer drug. See, for example, WO 88/07378 and U.S. Pat. No. 4,975,278.

The enzyme component of such conjugates includes any enzyme capable of acting on a prodrug in such a way so as to covert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic5-fluorocytosine into the anti-cancer drug, fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydratecleaving enzymes such as 13-galactosidase and neuraminidase useful for converting glycosylated prodrugs into free drugs; 13-lactamase useful for converting drugs derivatized with 13-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328:457-458 (1987)). Antagonist-abzyme conjugates can be prepared as described herein for delivery of the abzyme to a tumor cell population.

The enzymes of this invention can be covalently bound to the antagonist by techniques well known in the art such as the use of the heterobifunctional crosslinking reagents discussed above. Alternatively, fusion proteins comprising at least the antigen binding region of an antagonist of the invention linked to at least a functionally active portion of an enzyme of the invention can be constructed using recombinant DNA techniques well known in the art (see, e.g., Neuberger et al, Nature, 312:604-608 (1984)).

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol.

The antibodies disclosed herein may also be formulated as liposomes. Liposomes containing the antagonist are prepared by methods known in the art, such as described in Epstein et al., Proc. Natl. Acad. Sci. USA, 82:3688 (1985); Hwang et al., Proc. Natl Acad. Sci. USA, 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG derivatized phosphatidylethanolamine (PEG-PE). Liposomes are extruded through filters of defined pore size to yield liposomes with the desired diameter. Fab′ fragments of an antibody of the present invention can be conjugated to the liposomes as described in Martin et al. J. Biol. Chem. 257:286-288 (1982) via a disulfide interchange reaction. A chemotherapeutic agent is optionally contained within the liposome. See Gabizon et al. J. National Cancer Inst. 81(19)1484 (1989).

Amino acid sequence modification(s) of protein or peptide antagonists described herein are contemplated. For example, it may be desirable to improve the binding affinity and/or other biological properties of the antibody. Amino acid sequence variants of the antibody are prepared by introducing appropriate nucleotide changes into the antibody encoding nucleic acid, or by peptide synthesis. Such modifications include, for example, deletions from, and/or insertions into and/or substitutions of, residues within the amino acid sequences of the antagonist. Any combination of deletion, insertion, and substitution is made to arrive at the final construct, provided that the final construct possesses the desired characteristics. The amino acid changes also may alter post-translational processes of the antagonist, such as changing the number or position of glycosylation sites.

A useful method for identification of certain residues or regions of the antibody that are preferred locations for mutagenesis is called “alanine scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antagonist variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antagonist with an N-terminal methionyl residue or the antagonist fused to a cytotoxic polyp eptide. Other insertional variants of the antagonist molecule include the fusion to the N- or C-terminus of the antagonist of an enzyme, or a polypeptide which increases the serum half-life of the antagonist.

Another type of variant is an amino acid substitution variant. These variants have at least one amino acid residue in the antagonist molecule replaced by different residue. The sites of greatest interest for substitutional mutagenesis of antibody antagonists include the hypervariable regions, but FR alterations are also contemplated. Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened.

TABLE 1

| Original Residue | Exemplary Substitutions | Preferred Substitutions |
|---|---|---|
| Ala (A) | val; leu; ile | val |
| Arg (R) | lys; gin; asn | lys |
| Asn (N) | gln; his; asp, lys; arg | gln |
| Asp (D) | glu; asn | glu |
| Cys (C) | ser; ala | ser |
| Gln (Q | asn; glu | asn |
| Glu (E) | asp; gin | asp |
| Gly (G) | ala | ala |
| His (H) | asn; gin; lys; arg | arg |
| Ile (I) | leu; val; met; ala; | leu |
| | phe; norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophiuic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class.

Any cysteine residue not involved in maintaining the proper conformation of the antagonist also may be substituted, generally with serine, to improve the oxidative stability of the molecule and prevent aberrant crosslinking. Conversely, cysteine bonds) may be added to the antagonist to improve its stability (particularly where the antagonist is an antibody fragment such as an Fv fragment).

A particularly preferred type of substitutional variant involves substituting one or more hypervariable region residues of a parent antibody (e.g. a humanized or human antibody). Generally, the resulting variants selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identified hypervariable region residues contributing significantly to antigen binding. Alternatively, or in addition, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antagonist. By altering is meant deleting one or more carbohydrate moieties found in the antagonist, and/or adding one or more glycosylation sites that are not present in the antagonist.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly seine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more seine or threonine residues to the sequence of the original antagonist (for O-linked glycosylation sites).

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antagonist.

It may be desirable to modify the antibodies used in the invention to improve effector function, e.g. so as to enhance antigen-dependent cell-mediated cyotoxicity (ADCC) and/or complement dependent cytotoxicity (CDC) of the antagonist. This may be achieved by introducing one or more amino acid substitutions in an Fc region of an antibody antagonist. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989).

To increase the serum half life of the antibody, one may incorporate a salvage receptor binding epitope into the antagonist (especially an antibody fragment) as described in U.S. Pat. No. 5,739,277, for example. As used herein, the term “salvage receptor binding epitope” refers to an epitope of the Fc region of an IgG molecule (e.g., IgGI, IgG2, IgG3, or IgG4) that is responsible for increasing the in vivo serum half-life of the IgG molecule.

IV. Pharmaceutical Formulations

Therapeutic formulations comprising antagonists used in accordance with the present invention are prepared for storage by mixing an antagonist having the desired degree of purity with optional pharmaceutically acceptable carriers, excipients or stabilizers (Remington 's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parab ens such as methyl or propyl parab en; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEENTM, PLURONICSTM or polyethylene glycol (PEG).

The immunomodulatory antibody and the B cell depleting antibody may be in the same formulation or may be administered in difficult formulations. The composition may further include other non-antibody antagonists, e.g., CD40L or B7 antagonists. Examples there of include soluble CD40, B7 and fusions thereof. Administration can be concurrent or sequential, and may be effective in either order.

Exemplary anti-CD20 antibody formulations are described in WO98/56418, expressly incorporated herein by reference. This publication describes a liquid multidose formulation comprising 40 mg/mL rituximab, 25 mM acetate, 150 mM trehalose, 0.9% benzyl alcohol, 0.02% polysorbate 20 at pH 5.0 that has a minimum shelf life of two years storage at 2-8° C. Another anti-CD20 formulation of interest comprises 10 mg/mL rituximab in 9.0 mg/mL sodium chloride, 7.35 mg/mL sodium citrate dihydrate, 0.7 mg/niL polysorbate 80, and Sterile Water for Injection, pH 6.5.

Lyophilized formulations adapted for subcutaneous administration are described in WO97/04801 Such lyophilized formulations may be reconstituted with a suitable diluent to a high protein concentration and the reconstituted formulation may be administered subcutaneously to the mammal to be treated herein.

The formulation herein may also contain more than one active compound as necessary for the particular indication being treated, preferably those with complementary activities that do not adversely affect each other. For example, it may be desirable to further provide a chemotherapeutic agent, cytokine or immunosuppressive agent (e.g. one which acts on T cells, such as cyclosporin or an antibody that binds T cells, e.g. one which binds LFA-1). The effective amount of such other agents depends on the amount of antagonist present in the formulation, the type of disease or disorder or treatment, and other factors discussed above. These are generally used in the same dosages and with administration routes as used hereinbefore or about from 1 to 99% of the heretofore employed dosages.

The active ingredients may also be entrapped in microcapsules prepared, for example, by 30 coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington 's Pharmaceutical Sciences 16th edition, Osol, A. Ed. (1980).

Sustained-release preparations may be prepared. Suitable examples of sustained release preparations include semipermeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, noir degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. The formulations to be used for in vivo administration must be sterile. This is readily accomplished by filtration through sterile filtration membranes.

V. Treatment with the B Cell Depleting Antibody and Immunoregulatory Antibody

A composition comprising B cell depleting antibody and/or an immunoregulatory antibody will be formulated, dosed, and administered in a fashion consistent with good medical practice. Factors for consideration in this context include the particular B cell malignancy or disorder being treated, the particular mammal being treated, the clinical condition of the individual patient, the cause of the disease or disorder, the site of delivery of the agent, the method of administration, the scheduling of administration, and other factors known to medical practitioners. The therapeutically effective amount of the antagonist to be administered will be governed by such considerations.

As noted previously, the B cell depleting antibody and the immunoregulatory antibody may be in the same or in different formulations. These antagonist formulations can be administered separately or concurrently, and in either order. Preferably, the B cell depleting antibody specific to the B cell antigen target, e.g., CD20, CD19, CD22, CD37 or CD22, will be administered separately from the immunoregulatory antibody, e.g., an anti-CD40L antibody, anti-CD40 antibody, or anti-B7 antibody. Preferably, the CD40L antibody will be the humanized anti-CD40L antibody disclosed in U.S. Pat. No. 6,001,358 and the anti-B7 antibody the primatized antibody disclosed in U.S. Pat. No. 6,113,898. As noted, this antibody has recently been show to possess apoptotic activity. Also the preferred CD40L antibody has been shown to have efficacy in treatment of both T and B cell autoimmune diseases. Also, unlike another humanized anti-CD40L antibody (5c8) reported by Biogen, this antibody is not known to cause any adverse toxicity.

As a general proposition, the therapeutically effective amount of an antibody administered parenterally per dose will typically be in the range of about 0.1 to 500 mg/kg of patient body weight per day, with the typical initial range of antagonist used being in the range of about 2 to 100 mg/kg.

The preferred B cell depleting antibody is RITUXAN®. Suitable dosages for such antibody are, for example, in the range from about 20 mg/m2 to about 1000 mg/m2. The dosage of the antibody may be the same or different from that presently recommended for RITUXAN® for the treatment of non-Hodgkin's lymphoma. For example, one may administer to the patient one or more doses of substantially less than 375 mg/m2 of the antibody, e.g. where the dose is in the range from about 20 mg/m² to about 250 mg/m², for example from about 50 mg/m² to about 200 mg/m².

Moreover, one may administer one or more initial doses) of the antibody followed by one or more subsequent dose(s), wherein the mg/m² dose of the antibody in the subsequent doses) exceeds the mg/m² dose of the antibody in the initial dose(s). For example, the initial dose may be in the range from about 20 mg/m² to about 250 mg/m² (e.g. from about 50 mg/m² to about 200 mg/m²) and the subsequent dose may be in the range from about 250 mg/m² to about 1000 mg/m².

As noted above, however, these suggested amounts of both immunoregulatory and B cell depleting antibody are subject to a great deal of therapeutic discretion. The key factor in selecting an appropriate dose and scheduling is the result obtained, as indicated above. For example, relatively higher doses may be needed initially for the treatment of ongoing and acute diseases. To obtain the most efficacious results, depending on the particular B cell malignancy, the antagonist is administered as close to the first sign, diagnosis, appearance, or occurrence of the disease or disorder as possible or during remissions of the disease or disorder.

The antibodies are administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. In addition, the antibody may suitably be administered by pulse infusion, e.g., with declining doses of the antibody. Preferably the dosing is given by injections, most preferably intravenous or subcutaneous injections, depending in part on whether the administration is brief or chronic.

One additionally may administer other compounds, such as chemotherapeutic agents, immunosuppressive agents and/or cytokines with the antibodies herein. The combined administration includes co-administration, using separate formulations or a single pharmaceutical formulation, and consecutive administration in either order, wherein preferably there is a time period while both (or all) active agents simultaneously exert their biological activities.

Aside from administration of antibodies to the patient the present application contemplates administration of antibodies by gene therapy. Such administration of nucleic acid encoding the antibodies is encompassed by the expression “administering a therapeutically effective amount of an antagonist”. See, for example, WO96/07321 published Mar. 14, 1996 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antagonist is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAF-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno associated virus) and lipid-based systems (useful lipids for lipid-mediated transfer of the gene are DOTMA, DOPE and DC-Chol, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., .l. Biol. Chem 262:4429-4432 (1987); and Wagner et al., Proc. Natl. Acad. Sci. USA 87:3410-3414(1990). For review of the currently known gene marking and gene therapy protocols see Anderson et al., Science 256:808-813 (1992). See also WO 93/25673 and the references cited therein.

VI. Articles of Manufacture

In another embodiment of the invention, an article of manufacture containing materials useful for the treatment of the diseases or disorders described above is provided.

The article of manufacture comprises a container and a label or package insert on or associated with the container. Suitable containers include, for example, bottles, vials, syringes, etc. The containers may be formed from a variety of materials such as glass or plastic. The container holds or contains a composition which is effective for treating the disease or disorder of choice and may have a sterile access port (for example the container may be an intravenous solution bag or a vial having a stopper pierceable by a hypodermic injection needle). As whole, there may be one or several compositions. At least one active agent in one of those compositions is an antibody having B cell depleting activity and at least one antibody is an immunoregulatory antibody such as an anti-CD40L, anti-CD40, anti-CD23, anti-CD4 or anti-B7 antibody. The label or package insert indicates that the composition is used for treating a patient having or predisposed to B cell malignancy, such as those listed hereinabove. The article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Further details of the invention are illustrated by the following non-limiting Examples. The disclosures of all citations in the specification are expressly incorporated herein by reference.

The antibodies of the invention may be administered to a human or other animal in accordance with the aforementioned methods of treatment in an amount sufficient to produce such effect to a therapeutic or prophylactic degree. Such antibodies of the invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables.

The routine of administration of the antibody (or fragment thereof) of the invention may be oral, parenteral, by inhalation or topical. The term parenteral as used herein includes intravenous, intraperitoneal, intramuscular, subcutaneous, rectal or vaginal administration. The subcutaneous and intramuscular forms of parenteral administration are generally preferred.

The daily parenteral and oral dosage regimes for employing compounds of the invention to prophylactically or therapeutically induce immunosuppression, or to therapeutically treat carcinogenic tumors will generally be in the range of about 0.05 to 100, but preferably about 0.5 to 10, milligrams per kilogram body weight per day.

The antibodies of the invention may also be administered by inhalation. By “inhalation” is meant intranasal and oral inhalation administration. Appropriate dosage forms for such administration, such as an aerosol formulation or a metered dose inhaler, may be prepared by conventional techniques. The preferred dosage amount of a compound of the invention to be employed is generally within the range of about 10 to 100 milligrams.

The antibodies of the invention may also be administered topically. By topical administration is meant non-systemic administration and includes the application of an antibody (or fragment thereof) compound of the invention externally to the epidermis, to the buccal cavity and instillation of such an antibody into the ear, eye and nose, and where it does not significantly enter the blood stream. By systemic administration is meant oral, intravenous, intraperitoneal and intramuscular administration. The amount of an antibody required for therapeutic or prophylactic effect will, of course, vary with the antibody chosen, the nature and severity of the condition being treated and the animal.

### EXAMPLES

### Example 1

### Properties of B Lymphoma Cells. DHT-4 Cells

The concept that anti-CD40L antibody could block CD40L-CD40 mediated survival of malignant B-cells from chemotherapy induced toxicity/apoptosis was tested in vitro using IDEC-131, and the B-lymphoma cell line, DHL-4 (Roos et al., Leuk. Res. 10: 195-202 (1986)) exposed to adriamycin (ADM). IDEC-131 is a humanized version of the murine, monoclonal anti-human CD40L antibody, 24-31.

Initially, the minimum concentration of ADM cytotoxic to DHL-4 cells was determined by exposing DHL-4 cells for 4 hours to different concentrations of ADM. The cell cytotoxicity of DHL-4 cells after 5 days in culture was measured by Alamar Blue, a dye-reduction assay by live cells (see Gazzano-Santoro et al., J. Immunol. Meth. 202: 163-171 (1997)). Briefly, 1×10⁵ DHL-4 cells in growth medium (RMPI-1640 plus 10% Fetal Calf Serum) were incubated with varying concentrations of ADM (1×10⁻⁶ M to 1×10⁻⁸ M) in cell culture tubes at 37° C. for 4 hours. After incubation, cells were washed, re-suspended in growth medium at 1×10⁵ cells/ml concentration and 200 μl of cell suspension was added to each well of 96-well flat-bottom plate. Plates were incubated at 37° C. and tested for cytotoxicity at different time points. During the last 18 hours of incubation, 50 μl of redox dye Alamar Blue (Biosource International, Cat. #DAL 1100) was added to each well. Following incubation, plates were cooled by incubating at room temperature for 10 minutes on a shaker, and the intracellular reduction of the dye was determined. Fluorescence was read using a 96-well fluorometer with excitation at 530 nm and emission at 590 nm. The results are expressed as relative fluorescence units (RFU). The percentage of cytotoxicity was calculated as follows:

[1-(average RFU of test sample÷Average RFU of control cells)]×100%. Titration curve of ADM cytotoxicity was established and minimal concentrations of the drug for cytotoxicity was selected for subsequent assays.

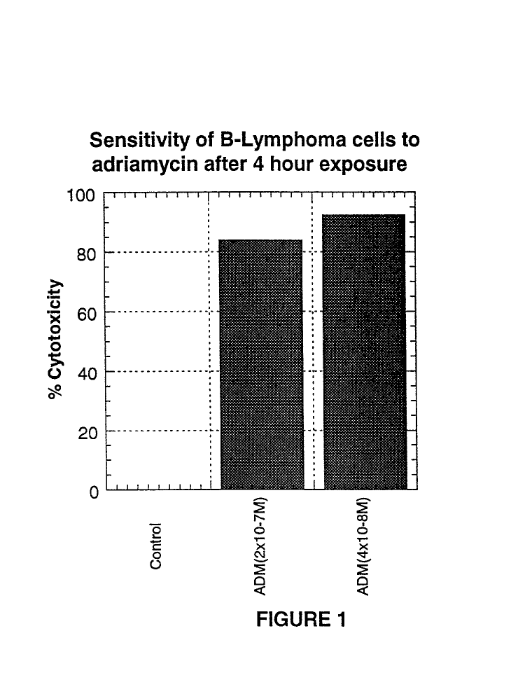

The results, as displayed in FIG. 1, shows cell cytotoxicity of DHL-4 cells cultured for 5 days after being exposed to ADM (2×10⁻⁷ M and 4×10⁻⁸ M of ADM) for 4 hours prior to culture. Cells were washed once after exposure and cultured in growth medium for 5 days and cytotoxicity determined by Alamar Blue dye-uptake assay, as described above. Additionally, the DHL-4 cells were characterized for the membrane expression of selected CD molecules by flow cytometry. DHL-4 cells have been found to express CD 19, CD20, CD40 molecules, but no expression of CD40L was detected.

### Example 2

### Anti-CD40L Antibody Overrides CD40L Mediated Resistance to Killing by to killing by Adriamy in of -lymphoma cells

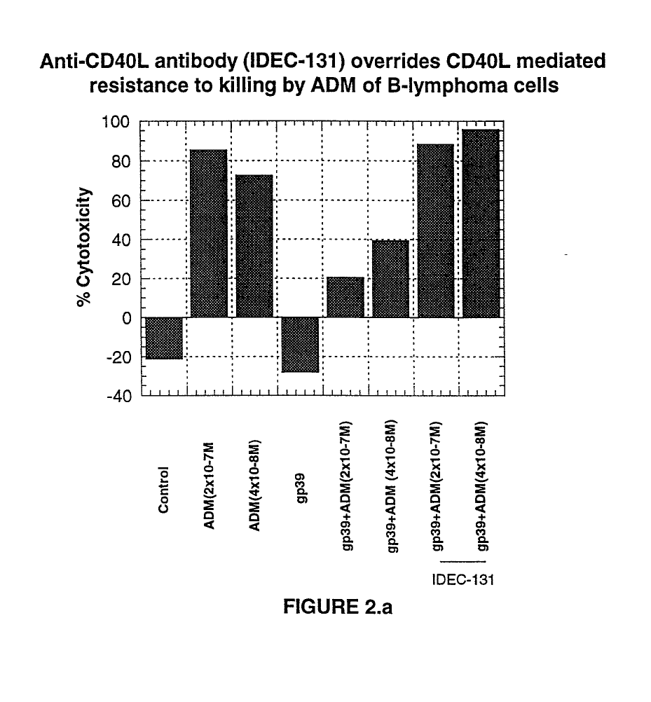

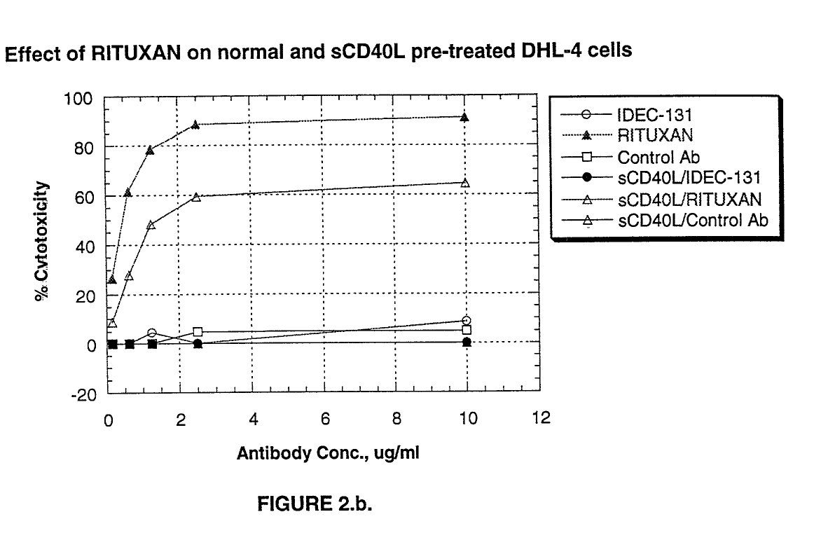

FIG. 2A shows the effect of an anti-CD40L antibody (IDEC-131) on CD40L-CD40 mediated resistance of DHL-4 cells to cell death induced by ADM. DHL-4 cells (0.5×10⁶ cells/ml) were incubated in the presence of 10 μg/ml of soluble CD40L (sCD40L, P. A. Brams, E. A. Padlan, K. Hariharan, K. Slater, J. Leonard, R. Noelle, and R. Newman, “A humanized anti-human CD 154 monoclonal antibody blocks CD 154-CD40 mediated human B cell activation,” (manuscript submitted)) for 1 hour at 37° C. After 1 hour of incubation, low concentrations of ADM (2×10⁻⁷ M−4×10⁻⁸ M) were added and incubated for another 4 hours in the presence or absence of CD40L (10 μg/ml). Following exposure to ADM, cells were washed and resuspended in growth medium at 0.5×10⁶ cells/ml concentration, and 100 μl of cell suspension added to each well of 96-well flat bottom plate, in duplicate, with or without sCD40L. sCD40L (10 μg/ml) was added to cultures that have been continuously exposed to sCD40L during ADM treatment and to cultures that had no sCD40L during ADM exposure. In addition, IDEC-131 at 10 μg/ml was added to cultures to determine its effect on DHL-4 cells incubated with sCD40L and ADM. After 5 days, the cytotoxicity was measured by Alamar Blue dye-uptake assay, as described.

Data show that sCD40L prolonged survival of DHL-4 cells after ADM treatment, whereas, as expected, increased cytotoxicity was observed in cells that were exposed to ADM in the absence of sCD40L. Furthermore, addition of anti-CD40L antibody (IDEC-131) reversed CD40L mediated cell survival, leading to increase in cell cytotoxicity (FIG. 2A).

The addition of IDEC-131 alone had no effect on DHL-4 cells treated with sCD40L, which indicates that the antibody, by itself, does not have any direct inhibitory or cytotoxic activities on DHL-4 cells (FIG. 2B). DHL-4 cells pre-incubated with and without sCD40L were cultured in the presence of different concentrations of IDEC-131, RITUXAN®, the anti-CD20 antibody CE9.1, and anti-CD4 antibodies (Anderson et al., Clin. Immunol. & Immunopathol. 84: 73-84 (1997)). After 5 days, the cytotoxicity/proliferation of DHL-4 cells was determined by Alamar Blue assay, as described above. FIG. 2B shows no effect on the proliferation or the cytotoxicity of DHL-4 cells by IDEC-131, whereas RITUNAN®, as expected, inhibited cell proliferation and induced cytotoxicity. No effect was seen in the DHL-4 cells cultured with anti-CD4 antibodies.

### Example 3

### CD40L-CD40 Signaling Prevents Apoptosis of B-lymphoma Cells by Anti-CD20 Antibody, RITUXAN®

The effect of CD40L-CD40 mediated signaling on anti-CD20 antibody induced apoptosis of B-lymphoma cells was determined using an in vitro system involving DHL-4 cells and the surface cross-linking of RITUXAN®. DHL-4 cells (0.5 to 1×10⁶ cells/ml) were cultured with sCD40L (10 μg/ml) at 37° C. After overnight culture, cells were harvested and incubated with 10 μg/ml of RITUXAN® or the control antibody (CE9.1; an anti-CD4 antibody) with or without sCD40L (10 μg/ml) on ice. After 1 hour of incubation, cells were centrifuged to remove unbound antibodies, and resuspended at 1×10⁶ cells/ml in growth medium (5% FCS-RPMI) and cultured in tissue culture tubes. The cells surface bound antibodies were cross-linked by spiking F(ab′)₂ fragments of goat anti-human Ig-Fcγ specific antibodies at 15 μg/ml, and the cultures were incubated at 37° C. until assayed for apoptosis. Apoptosis was detected using a flow cytometry caspase-3 assay. Cultured cells were harvested at 4 and 24 hours, washed and fixed at 4° C. using Cytofix (Cytofix/Cytopern™ Kit, Pharmingen Cat. #2075KK). After 20 min of fixation, cells were washed and 15 μl of affinity purified PE-conjugated polyclonal rabbit anti-caspase-3 antibody (Pharmingen, Cat. # 67345) and 50 μl of cytoperm (Pharmingen; Cat. #2075KK) were added. Cells were incubated on ice in the dark for 30 min. After incubation cells were washed once and resuspended in cytoperm. Flow cytometry data was acquired on FACScan and analyzed using WinList software from Verity Software House.

Table I shows resistance of RITUXAN® induced apoptosis in DHL-4 lymphoma cells by exposure to sCD40L. In these studies, activation of caspase-3 was used as the surrogate marker since our previous studies revealed good correlation between caspase-3 and Tunel assay. Cross-linking of RITUXAN® on the DHL-4 cell surface in the presence of sCD40L decreased levels of apoptosis, whereas cells not exposed to sCD40L apoptosed. In comparison, cultures incubated in the presence of an antibody of the same isotype, control antibody (CE9.1), resulted in no apoptosis of the cells. Thus, the data suggests that sCD40L induced signaling of CD40 pathway can lead to development of RITUXAN® mediated killing of B-lymphoma cells.

TABLE I
Resistance of RITUXAIN0 mediated apoptosis of DHL-4 cells by sCD40L

| | % Apoptosis (IVHF)^{(a)} | |
|---|---|---|
| Culture Conditions | 4 Hours | 24 Hours |
| DHT-4 cells exposed to sCD40L | | |
| Cells only | 3.35 (17.42) | 4.94 (7.62) |
| Cells + RITUXAN | 1.97 (1.97) | 4.54 (6.54) |
| Cells + RITUXAN + | 21.17 (17.39) | 9.62 (13.44) |
| anti-hu.IgG.F(ab′)₂ | | |
| Cells + CE9.1 | 2.31 (13.25) | 4.15 (7.85) |
| Cells + CE9.1 + anti-hu.IgG.F(ab′)₂ | 2.09 (22.14) | 4.14 (9.57) |
| Cells + anti-hu.IgG.F(ab′)₂ | 1.93 (12.57) | 5.13 (8.02) |
| DHL-4 cells not exposed to sCD40L | | |
| Cells only | 4.36 (14.34) | 5.08 (17.62) |
| Cells + RITUXAN | 5.67 (10.66) | 1.08 (17.92) |
| Cells + RITUXAN + | 74.82 (22.80) | 30.63 (26.84) |
| anti-hu.IgG.F(ab′)₂ | | |
| Cells + CE9.1 | 5.99 (14.00) | 3.05 (18.24) |
| Cells + CE9.1 + anti-hu.I-G.F(ab′)₂ | 5.96 (12.11) | 2.24 (18.19) |
| Cells + anti-hu.IgG.F(ab′)₂ | 6.09 (12.27) | 1.85 (17.27) |

^{(a)}Percent positive cells with caspase-3 activity and its mean fluorescent intensity in log scale.

### Example 4

### Effect of IDEC-131 on the Survival of chronic Lymphocytic Leukemia (CLL) Cells

To determine the effect of IDEC-131 on the growth and survival of B-CLL cells in vitro, B-CLL cells were cultured with and without IDEC-131 in the presence of CD40L in vitro. Peripheral blood mononuclear cells (PBMC) were isolated from a CLL patient's blood using a Ficoll-Hypaque gradient centrifugation. Viability was determined by Trypan blue dye exclusion and was >98%. Flow cytometric analysis revealed that >70% of the lymphocytes were CD 19⁺/CD20⁺. CLL cells (PBMC) were cultured in CLL growth medium (e.g., RPMI-1640 medium supplemented with 5% FCS or 2% of autologous donor plasma, supplemented with 2 mM L-Glutamine and 100 U/ml Penicillin-Streptomycin). In addition, for some experiments, CD19⁺ B-cells were purified using CD19⁺ Dynabeads™ as per manufacture's instructions (Dynal, Cat. #111.03/111.04) and cultured as above. CLL or purified B-CLL cells cultured in growth medium mostly under went spontaneous apoptotic cell death. However, culturing these cells in the presence of sCD40L extended their viability in cultures. Table II indicates the cell viability of CD 19⁺ B-CLL cells grown in the presence or absence of sCD40L (5 μg/ml) at different time points and indicates the longer survival of CLL cells. B-CLL cells from Patient #1 cultured with sCD40L had ≧60% viability for greater than 2 weeks, whereas cells grown in the absence of sCD40L had less than 10% viability.

TABLE II
Survival of B-CLL cells in the presence of sCD40L

| | | |
|---|---|---|
| B-CLL | Time | % Viability^{(a)} |

| Sample | (Hours) | (−) CD40L | (+) CD40L |
|---|---|---|---|
| Patient #1 | 0 | ≧90 | ≧90 |
| | 48 | 88 | 90 |
| | 96 | 46 | 77 |
| | 144 | 30 | 72 |
| Patient #2 | 0 | ≧90 | ≧90 |
| | 72 | 40 | 72 |
| | 96 | 31 | 65 |
| | 144 | 17 | 51 |

^{(a)}equals the percent viability determined by Trypan blue dye exclusion.

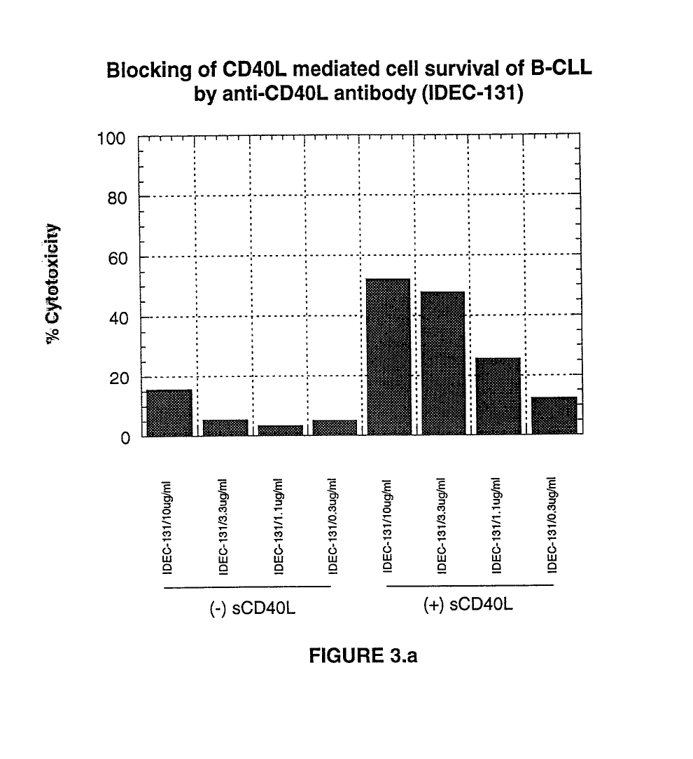

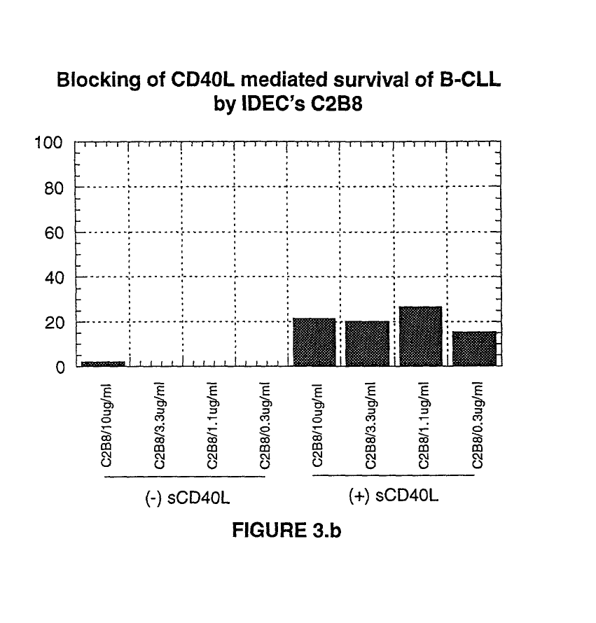

FIG. 3A shows the effect of IDEC-131 on the growth and survival of B-CLL cells after 7 days in culture. Purified B-CLL cells from a CLL patient (2×10⁶ cells/ml) were divided into two culture tubes. Cells in one tube were mixed with sCD40L (5 μg/ml) in equal volume of growth medium, whereas the other tube was incubated with equal volume of growth medium as control. After 1 hour of incubation at 37° C., cells were gently mixed and 100 μl of cell suspension media added to each well of a 96-well flat bottom plate in duplicate with and without varying concentrations of IDEC-131 (10 μg/ml to 0.3 μg/ml). Seven days later, cell survival/death in culture was determined by Alamar Blue assay, as described above. Data showed cell survival in cultures with sCD40L. The addition of IDEC-131 into culture resulted in increased cell death, which indicated a reversal of cell survival or a sensitization to cell death. Additionally, RITUXAN® administered at the same concentration as the IDEC-131 produced less of lower effect than IDEC-131 on cell death (FIG. 3B).

### Example 5

### CD40L-CD40 Mediated Up-regulation of HLA-DR Molecules in B-CLL

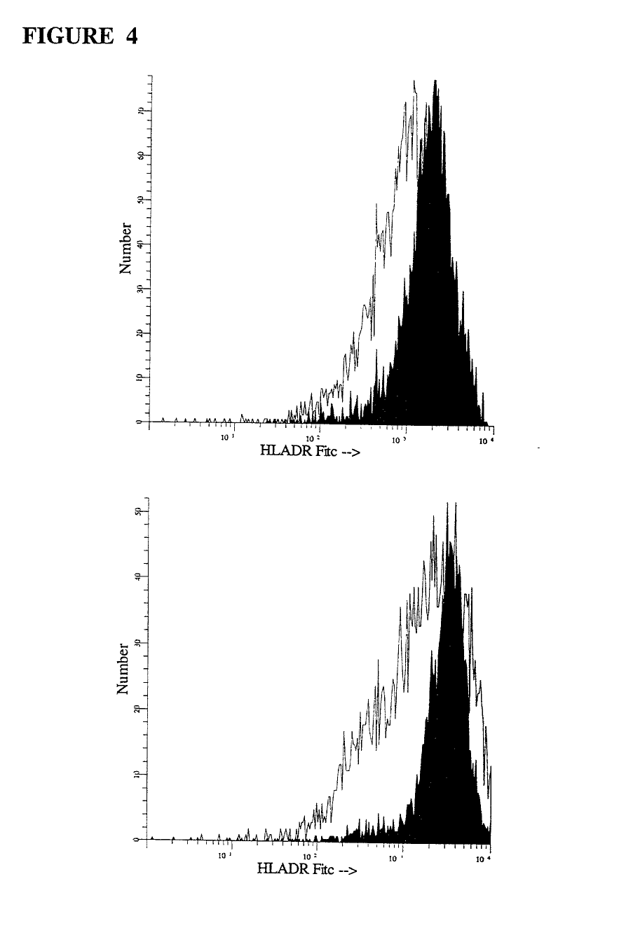

To determine whether the CD40L-CD40 signal transduction pathway is intact, CLL cells from CLL patients were cultured (5×10⁵ cells/ml) with and without 5 μg/ml of CD40L at 37° C. At 48 hours and 144 hours, the class II molecule, HLA-DR expression, was determined on CD 19⁺ cells by flow cytometry using standard procedures. Briefly, cultured lymphocytes were harvested at different time points and analyzed for surface expression of molecules using antibodies coupled to either fluorescein (FITC) or phycoerythrin (PE) for single or double staining using a FACScan (Becton-Dickinson) flow cytometer. To stain for flow cytometry, 1×10⁶ cells in culture tubes were incubated with appropriate antibodies as follows: anti-CD45-FITC to gate lymphocyte population on a scatter plot; anti-CD19-PE (Pharmingen, Cat. # 30655) or anti-CD20-FITC (Pharmingen; Cat. #33264) antibodies to determine the CD19⁺ and/or CD20⁺ B-cells; anti-CD3-FITC antibodies (Pharmingen; Cat. #30104) to gate-off the T cells; anti-CD 19-RPE and anti-HLA-DR-FITC antibodies (Pharmingen; Cat. #32384) to determine the Pclass II expression on CD 19⁺ cells. Cells were washed once by centrifugation (at 200×g, for 6 min.) with 2 ml cold PBS and incubated with antibody for 30 min. on ice, after which the cells washed once, fixed in 0.5% paraformaldehyde and stored at 4° C. until analyzed. Flow cytometry data was acquired on FACsan and analyzed using WinList software (Verity Software House). The machine was set to autogating to allow examination of quadrants containing cells that were single stained with either RPE or FITC, unstained or doubly stained. FIG. 4 shows the comparison of HLA-DR expression in CD 19⁺ CLL cells cultured with sCD40L and those cells not cultured with sCD40L. A higher level of HLA-DR expression was detected on B-CLL cells cultured in the presence of sCD40L (Table III).

TABLE III
CD40L-CD40 mediated up-regulation of HLA-DR molecule in B-CLL

HLA-DR^{+(a)}

| Sample | Time | % Positive | MFI |
|---|---|---|---|
| Control | 48 hrs | 81 | 92 |
| | 144 hrs | 88 | 1655 |
| Cells + sCD40L | 48 hrs | 88 | 101 |
| | 144 hrs | 95 | 2943 |

^{(a)}CD19⁺ B-cells that are positive for HLA-DR molecules and its mean fluorescent intensity (MIF).

### Example 6

### Preparation of IDEC-131 and RITUXAN®

For treatment of a CD40⁺ malignancy, IDEC-131 at about 10 to about 50 mg/ml in a formulation buffer 10 mM Na-citrate, 150 mM NaCl, 0.02% Polysorbate 80 at pH 6.5 is infused intravenously (iv) to a subject. IDEC-131 is administered before, after or in conjunction with RITUXAN®. The RITUXAN® dosage infused ranges from about 3 to about 10 mg/kg of subject weight.

### Example 7

### Preparation of IDEC-131 and CHOP

For treatment of CD40⁺ malignancies responsive to CHOP (e.g., Hodgkin's Disease, Non-Hodgkin's lymphoma and chronic lymphocytic leukemia, as well as salvage therapy for malignancies wherein cells are CD40⁺), IDEC-131 is infused at a dosage ranging from about 3 to about 10 mg per kg of patient weight immediately prior to the initiation of the CHOP cycle. IDEC-131 administration will be repeated prior to each CHOP cycle for a total of 4 to 8 cycles.

### Example 8

### Administration of anti-CD40L or anti-B7 in Combination with RITUXAN® to Treat B-cell Lymphoma in a Subject

Combination therapies are particularly useful as salvage therapies or for treating relapsed or aggressive forms of CD40⁺ malignancies (e.g., Hodgkin's Disease, Non-Hodgkin's lymphoma and CLL). When IDEC-131 is to be administered in combination with CHOP and RITUXAN®, IDEC-131 is administered as discussed above in Example 6, followed by the schedule specified for CHOP-IDEC-131 administration in Example 7. Alternatively, the same regimen is effected wherein IDEC-131 (anti-CD40L) is substantially within an anti-B7 antibody.

All references discussed above are hereby incorporated by reference in their entirety.

1. A method of treating a B cell malignancy in a subject in need of such treatment comprising administering a therapeutically effective amount of at least one immunoregulating or immunomodulating antibody that is selected from the group consisting of an anti-CD23, anti-B7, anti-CD40, anti-CD40L and anti-CD4 antibody and at least B cell depleting antibody selected from the group consisting of an anti-CD19, anti-CD20, anti-CD22 and anti-CD37 antibody, wherein said antibody administration is effected separately, in combination, and in either order of administration.

2. The method of claim 1 wherein B cell malignancy is non-Hodgkin's lymphoma.

3. The method of claim 1 wherein the B cell malignancy is B cell lymphoma.

4. The method of claim 1 wherein the B cell malignancy is a leukemia.

5. The method of claim 1 wherein treatment comprises the administration of an anti-B7 antibody and an anti-CD20 antibody.

6. The method of claim 5 wherein the anti-CD20 is RITUXAN®.

7. The method of claim 5 wherein the anti-B7 antibody is a Primatized antibody.

8. The method of claim 7 wherein the anti-B7 induces apoptosis of cancer cells.

9. The method of claim 1 wherein the immunoregulatory antibody is administered after the B cell depleting antibody.

10. The method of claim 1 wherein the immunoregulatory antibody is administered before the B cell depleting antibody.

11. The method of claim 1 wherein the B cell depleting antibody and the immunoregulatory antibody are administered within about a month of each other.

12. The method of claim 1 wherein the B cell depleting antibody and the immunoregulatory antibody are administered within about one week of each other.

13. The method of claim 1 wherein the B cell depleting antibody and the imunoregulatory antibody are administered within about 1 day of each other.

14. The method of claim 1 wherein is used to treat a B cell malignancy selected from the group consisting of relapsed Hodgkin's disease, resistant Hodgkin's disease high grade, low grade and intermediate grade non-Hodgkin's lymphomas, small lymphocytic/B cell chronic lymphocytic leukemia (SLL/B-CLL), lymhoplasmacytoid lymphoma (LPL), mantle cell lymphoma (MCL), follicular lymphoma (FL), diffuse large cell lymphoma (DLCL), Burkitt's lymphoma (BL), AIDS-related lymphomas, monocytic B cell lymphoma, angioimmunoblastic lymphoadenopathy, small lymphocytic; follicular, diffuse large cell; diffuse small cleaved cell; large cell immunoblastic lymphoblastoma; small, non-cleaved; Burkitt's and non-Burkitt's; follicular, predominantly large cell; follicular, predominantly small cleaved cell; and follicular, mixed small cleaved and large cell lymphomas.

15. The method of claim 14 wherein said B cell malignancy is Hodgkin's disease.

16. The method of claim 1 wherein either or both antibody is attached to a radiolobel.

17. The method of claim 1 which further comprises chemotherapy or radiation therapy.

18. The method of claim 1 which includes administration of a non-antibody antagonist specific to CD40L or B7.
